(19) United States
(12) Patent Application Publication
(10) Pub. No.: US 2022/0273835 A1
(43) Pub. Date: Sep. 1, 2022

(54) QUICK HAND SANITIZING METHOD AND QUICK HAND SANITIZING DEVICE

(71) Applicants: M&C Design Co., Ltd., Kawasaki-shi, Kanagawa (JP); UNIVERSITY OF FUKUI, Fukui-shi, Fukui (JP)

(72) Inventors: Hiromichi Iwasaki, Yoshida-gun (JP); Eiji Tanaka, Kawasaki-shi (JP); Yukio Hida, Yoshida-gun (JP); Nobuo Bessho, Kawasaki-shi (JP)

(73) Assignees: M&C Design Co., Ltd., Kawasaki-shi, Kanagawa (JP); UNIVERSITY OF FUKUI, Fukui-shi, Fukui (JP)

(21) Appl. No.: 17/608,906

(22) PCT Filed: May 1, 2020
(86) PCT No.: PCT/JP2020/018423

§ 371 Date: Nov. 4, 2021

(30) Foreign Application Priority Data
May 7, 2019 (JP) 2019-087933

(51) Int. Cl. A61L 2/10; A61L 2/18

(52) U.S. Cl. CPC A61L 2/10; A61L 2/18; A61L 2202/12; A61L 2202/20

(57) ABSTRACT

[Problem] To provide a method and a device for sanitizing hands quickly and easily in view of the current state of hand hygiene.

[Solution] A sanitization method which is characterized by involving both irradiation of ultraviolet light of a wavelength of between 190 to 230 nm and a treatment using an alcoholic sanitizer; a sanitization method which is characterized in that the alcoholic sanitizer comprises water and an alcoholic agent selected from any one of ethanol, propanol, or a mixture thereof, and the amount of alcoholic agent is greater than the amount of water; and a sanitization device which includes a sanitizer supply unit for depositing the alcoholic sanitizer on an object to be sanitized and an ultraviolet irradiation unit that irradiates the object to be sanitized with ultraviolet light of a wavelength of between 190 to 230 nm.

### TECHNICAL FIELD

The present invention relates to sanitizing method and device capable of disinfecting and sanitizing a part of a human body (mainly hands) and various products and tools simply and conveniently with high disinfecting power.

### BACKGROUND ART

Conventionally, alcohol-based sanitizers have been used as standard sanitization for a human body such as hands, wherein sanitizing for 20 to 30 seconds is recommended. However, the rates of compliance with hand sanitization among nurses and the like at busy hospitals are low. (NPL 1) On the other hand, various techniques for sanitizing an object using the bactericidal effect of ultraviolet light have been known. However, since ultraviolet light is harmful to humans, there are many restrictions on the use of ultraviolet light, such as the amount of radiation and irradiation places.

However, in recent years, it has been found that ultraviolet light of 230 nm or less has little effect on humans and exerts a high bactericidal effect (NPL 2, and the like), and a plurality of proposals applying this result have been made.

For example, PTL 1 to PTL 3 and PTL 5 each propose a sanitizing device using ultraviolet light of 230 nm or less.

Also, PTL 4 proposes a sanitizing method using a combination of ultraviolet light and a disinfectant.

### CITATION LIST

### Non Patent Literature

[NPL 1] WHO Guidelines on Hand Hygiene in Health Care, Summary whqlibdoc.who.int/hq/2009/WHO_IER_PSP_2009.07_eng.pdf

[NPL 2] Germicidal Efficacy and Mammalian Skin Safety of 222-nm UV Light Radiat Res. 2017 April; 187 (4): 483-491

### Patent Literature

[PTL 1] Japanese Laid-Open Patent Publication No. 2018-114197

[PTL 2] Japanese Laid-Open Patent Publication No. 2016-220684

[PTL 3] Japanese Unexamined Patent Application Publication (Translation of PCT Application) No. 2014-508612

[PTL 4] Japanese Laid-Open Patent Publication No. 2007-82900

[PTL 5] Japanese Laid-Open Patent Publication No. 2017-136145

### SUMMARY OF INVENTION

### Technical Problem

Unfortunately, a problem with the proposed sanitizing method described above using an alcohol-based sanitizer and the sanitizing method and device according to NPL 1, PTL 1 to PTL 3 and PTL 5 is that sanitization still takes 15 seconds or longer. The proposal according to PTL 4 not only uses 254 nm ultraviolet light which is harmful to humans, but also uses an aqueous solution containing peroxycarboxylic acid as the sanitizer, which causes a skin problem when used on a human body.

In other words, the conventional proposals have not yet resolved the problems related to quick sanitization and minimizing the impact on humans such as skin problems; thus, a proposal solving these problems is desired under the current circumstances.

Therefore, an object of the present invention is to provide a sanitizing method and a sanitizing device that are capable of easily sanitizing an object to be sanitized, such as a hand, within a short period of time, and have little adverse effect on humans such as skin problems.

### Solution to Problem

As a result of diligent studies to solve the problems described, the inventors of the present invention have found that the foregoing object can be achieved by combining ultraviolet light of a certain wavelength and a certain sanitizer, and completed the present invention.

The present invention provides each of the following inventions.

1. A sanitizing method, comprising a step of executing both radiation of ultraviolet light having a wavelength of 190 nm to 230 nm and a treatment with an alcohol-based sanitizer.

2. The sanitizing method according to 1 above, wherein the alcohol-based sanitizer contains water and an alcoholic agent selected from among ethanol, propanol, and a mixture thereof, the alcoholic agent being blended in a greater amount than the water.

3. The sanitizing method according to 1 or 2 above, wherein the ultraviolet light has a wavelength of 206 to 208 nm or 221 to 223 nm.

4. The sanitizing method according to any one of 1 to 3 above, comprising a step of executing an alcohol-based sanitizer treatment in which a deposition amount of the alcohol-based sanitizer on an object to be sanitized is 0.2 mg/cm² to 2 mg/cm², and a step of setting an amount of ultraviolet radiation to 0.2 mJ/cm² to 5 mJ/cm².

5. A sanitizing device, comprising a sanitizer supply unit for depositing an alcohol-based sanitizer onto an object to be sanitized, and an ultraviolet irradiation unit for irradiating the object to be sanitized with ultraviolet light having a wavelength of 190 nm to 230 nm.

6. The sanitizing device according to 5 above, wherein the ultraviolet light has a wavelength of 206 to 208 nm or 221 to 223 nm.

7. The sanitizing device according to 5 or 6 above, wherein the sanitizer supply unit sprays the alcohol-based sanitizer in an amount at which a deposition amount of the alcohol-based sanitizer on the object to be sanitized is 0.2 mg/cm² to 2 mg/cm², and an amount of ultraviolet radiation is 0.2 mJ/cm² to 5 mJ/cm².

8. The sanitizing device according to any one of 5 to 7 above, further comprising an insertion space for the object to be sanitized, wherein the sanitizer supply unit is configured to be capable of spraying the alcohol-based sanitizer into the insertion space for the object to be sanitized, the ultraviolet irradiation unit is configured to be capable of irradiating the inside of the insertion space for the object to be sanitized, the insertion space for the object to be sanitized being composed of a housing, a part of which has an opening, and being configured as an independent space so that the ultraviolet irradiation unit and an energizer that supplies power to the sanitizer supply unit and the ultraviolet irradiation unit are not exposed into the space, preventing components of the alcohol-based sanitizer to be sprayed from entering the ultraviolet irradiation unit and the energizer. 9. The sanitizing device according to 8 above, wherein the opening has an area of 200 cm² to 450 cm².

10. The sanitizing device according to any one of 5 to 9 above, wherein at least the insertion space for the object to be sanitized partially has a wall surface made of a reflective material that reflects ultraviolet light having a wavelength of 222 nm.

### Advantageous Effects of Invention

According to the present invention, the sanitizing method and the sanitizing device thereof can sanitize an object to be sanitized, such as a hand, within a short period of time, and have little adverse effect on humans such as skin problems.

Therefore, the sanitizing method and the sanitizing device of the present invention can sanitize the hands of healthcare professionals in a short period of time for the purpose of hygienic hand hygiene (sanitization) of the healthcare professionals.

### BRIEF DESCRIPTION OF DRAWINGS

FIG. 1 is a perspective view, schematically showing a hand sanitizing device.

FIG. 2 is a cross-sectional view, schematically showing the hand sanitizing device of FIG. 1, taken along cross section II-II.

FIG. 3 is a cross-sectional view, schematically showing the hand sanitizing device of FIG. 1, taken along cross section

### REFERENCE SIGNS LIST

1 Sanitizing device

2 Chamber

3 UV lamp

4 Sanitizer tank

5 Pipe

6 Hand

7 Spray nozzle

### DESCRIPTION OF EMBODIMENTS

Embodiments of the present invention are now described hereinafter more specifically, but the present invention is not limited thereto.

First, the sanitizing method of the present invention is described hereinafter.

The sanitizing method of the present invention executes both radiation of ultraviolet light having a wavelength of 190 nm to 230 nm and a treatment with an alcohol-based sanitizer.

Examples of an object to be sanitized in the sanitizing method of the present invention include hands of humans and various devices and equipment, especially hands of healthcare professionals such as doctors and nurses at hospitals. Here, “hands” refer to the hands, fingers, wrists, and a section of the forearm near the wrist. Examples of the devices and equipment include surgical tools, treatment tools, gloves, and various other equipment and tools.

### Ultraviolet Light

The ultraviolet light radiated in the sanitizing method of the present invention is ultraviolet light with a wavelength of 190 to 230 nm. Here, “the ultraviolet light with a wavelength” is ultraviolet light whose main component is the ultraviolet light with the aforementioned wavelengths, and may include ultraviolet light other than the ultraviolet light with the aforementioned wavelengths. In such a case, the content of ultraviolet light with a wavelength equal to or greater than 230 nm is preferably 15% or less in terms of energy in the entire ultraviolet light. The wavelength distribution of the ultraviolet light is not particularly limited as long as the ultraviolet light has a wavelength in the range of 190 to 230 nm, and may exhibit a sharp peak with a half width of 2 nm at a specific wavelength or may have a wide spectral distribution. It is preferred that the range of wavelengths of the ultraviolet light be 200 to 225 nm, more preferably 206 to 208 (207) nm or 221 to 223 (222) nm, and more preferably 221 to 223 (222) nm.

An excimer lamp, an LED (Light emitting diode) or the like can be used as a lamp that emits ultraviolet light having a wavelength of 190 nm to 230 nm. When an excimer lamp containing KrBr or KrCl is used as the light source, since the emitted light often contains components with wavelengths longer than 230 nm, a bandpass filter or the like is preferably used to prevent the emission of light having a wavelength longer than 230 nm. Alternatively, it is preferred that the contained light having a wavelength longer than 230 nm be emitted in an amount not affecting the human body. As the ultraviolet light, ultraviolet light having a peak wavelength of 206 to 208 (207) nm (an excimer lamp containing KrBr) or 221 to 223 (222) nm (an excimer lamp containing KrCl) is preferably used in terms of the actual availability, and most preferably, ultraviolet light of a KrCl-containing excimer lamp having a peak wavelength of 221 to 223 (222) nm is used. In addition, ultraviolet LEDs such as diamond LEDs that emit light having these wavelengths are used. As a UV lamp using an excimer lamp, the one described in, for example, PTL 1, 2, 3 or 5 can be used, but examples of the UV lamp are not limited thereto.

### Alcohol-Based Sanitizer

Examples of the alcohol-based sanitizer are as follows.

A quick-drying alcohol-based sanitizer that mainly contains an aqueous solution of ethanol, propanol, or a mixture of both. An iodine-based sanitizer that is composed mainly of an alcoholic solution of iodine (iodine tincture) and an alcoholic solution of povidone iodine (a compound of polyvinylpyrrolidone and iodine); a quaternary ammonium salt-based sanitizer that is composed mainly of an alcoholic solution of benzalkonium chloride and benzethonium chloride; an amphoteric surfactant sanitizer that is composed mainly of an alcoholic solution of alkyldiaminoethylglycine hydrochlorid; and a biguanide sanitizer that is composed mainly of an alcoholic solution of chlorhexidine gluconate.

From the perspective of sanitization power, safety, quick-drying properties, and not requiring water or drainage facilities, a quick-drying alcohol-based sanitizer that mainly contains an aqueous solution of ethanol, propanol or a mixture thereof is most preferable. The blending ratio of water and alcohol in this aqueous solution is such that the alcohol content is preferably 50% by volume or more, and more preferably 60% by volume or more. Also, the alcohol content is preferably 95% by volume or less, and more preferably 85% by volume or less. A low blending ratio of the alcohol is unlikely to achieve a sufficient bactericidal/sanitization effect, and the aqueous solution might take longer to dry after application. On the other hand, a high blending ratio of the alcohol is also unlikely to achieve the sufficient bactericidal/sanitization effect, and the aqueous solution might get dry too quickly and tend to cause an intense skin irritation. When safety such as low ignitability is important, the alcohol concentration is preferably 50% by volume to 60% by volume. The alcohol-based sanitizer described above may contain a sanitizer containing a small amount of benzalkonium chloride, benzethonium chloride, and chlorhexidine gluconate, and if necessary, a small amount of a thickener such as carboxyvinyl polymer or cellulose-based water-soluble polymer compound. The alcohol-based sanitizer can also contain a moisturizer or the like to prevent chapped hands.

### UV Radiation and Treatment with Alcohol-Based Sanitizer

When treating with the alcohol-based sanitizer, it is preferred that the alcohol-based sanitizer be sprayed onto hands This allows the sanitizer to reach the under the nails (fingertip or hyponychium), the wrinkles of the hands, and deep into fingerprints without rubbing the hands together, thereby achieving the desired effects of the present invention more efficiently. Furthermore, the size of mist droplets is important in the sense that the sanitizer is deposited onto the hands without obstructing the supply of the ultraviolet light. When the treatment is conducted by spraying the sanitizer in this manner, the particle size of the mist droplets is preferably 500 μm or less on average, and more preferably 200 μam or less. The spraying method is not particularly limited as long as such average particle size can be achieved, and the spraying method can be conducted by a method using an ultrasonic sprayer, an air spray method, an airless spray method, or the like. Note that the average particle size of the mist droplets is the average of the largest diameter portions of granular mist droplets, and can be measured by a particle size measurement method such as a JIS Z 8825 laser diffraction/scattering method.

The method of sanitization by combining ultraviolet radiation and sanitizer treatment is not particularly limited. Any of a method of depositing the sanitizer onto an object to be sanitized and then irradiating said object with ultraviolet light, a method of irradiating said object with ultraviolet light at the same time as the deposition of the sanitizer (sanitizer treatment), and a method of performing the sanitizer treatment after irradiating said object with ultraviolet light, can be used. In particular, it is preferred that ultraviolet radiation and sanitizer treatment be executed simultaneously. Here, “simultaneously” means that the spraying by a device that sprays the sanitizer and the start of the radiation by a device emitting ultraviolet light are controlled by a normal control circuit or control mechanism so as to be executed simultaneously. Therefore, even if there exists an error equivalent to approximately a slight difference (within 1 to 3 seconds) caused by a normal device, such error falls within the meaning of “simultaneously.”

Additionally, from the perspective of achieving the desired effects of the present invention, it is particularly preferred that the sanitizer treatment described above be performed by spraying or applying the alcohol-based sanitizer onto the object to be sanitized in such a manner that the deposition amount of the alcohol-based sanitizer deposited onto the object to be sanitized is 0.2 mg/cm² to 2 mg/cm², and the amount of ultraviolet radiation is 0.2 mJ/cm² to 5 mJ/cm². Here, the deposition amount of the alcohol-based sanitizer can be realized by spraying a predetermined amount of the sanitizer into a space having a predetermined spatial volume, by using a device or the like described hereinafter.

The sanitizing method of the present invention can achieve the desired effects of the present invention more efficiently by using a specific sanitizing device.

Hereinafter, one embodiment of the sanitizing device of the present invention that can be favorably used in the sanitizing method of the present invention will be described with reference to the drawings, but the sanitizing device of the present invention is not limited thereto.

### Sanitizing Device

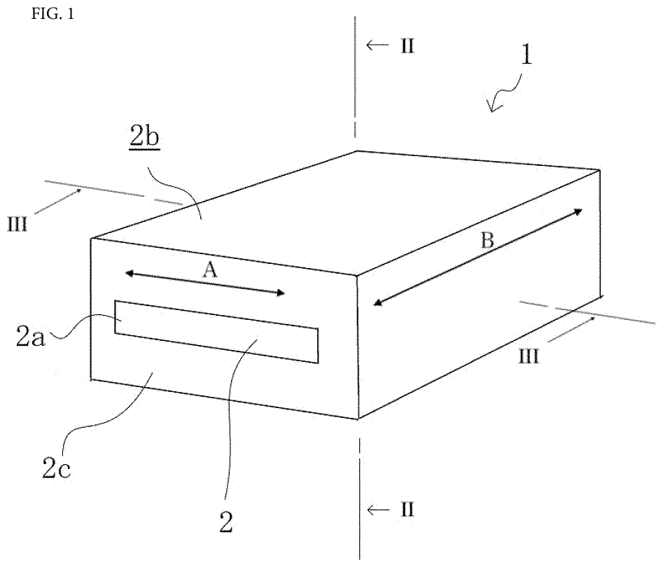

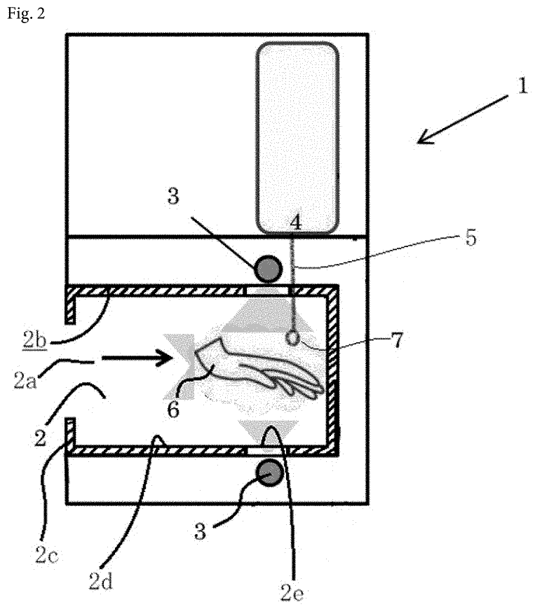

A sanitizing device 1 of the present invention shown in FIGS. 1 and 2 includes a sanitizer supply unit that deposits the alcohol-based sanitizer onto the object to be sanitized, and an ultraviolet irradiation unit that irradiates the object to be sanitized with ultraviolet light having a wavelength of 190 nm to 230 nm.

### Overall Structure

The sanitizing device 1 of the present embodiment shown in FIGS. 1 and 2 includes a housing 2b that has an opening 2a that forms an insertion space 2 for an object to be sanitized, which is formed to allow the insertion of the object to be sanitized (a hand 6 of a human, in the present embodiment), UV lamps 3 as ultraviolet irradiation units that are provided above and below the housing 2b, a sanitizer tank 4 for retaining the sanitizer, and a sanitizer spray nozzle 7 connected to the sanitizer tank 4 via a pipe 5, the sanitizer tank 4 and the spray nozzle 7 each functioning as the sanitizer supply unit.

### Sanitizer Supply Unit

The sanitizer supply unit is configured to be able to spray the alcohol-based sanitizer into the insertion space for the object to be sanitized, and is provided with the spray nozzle 7 so as to be able to spray the alcohol-based sanitizer. A commercially available spray nozzle can be used as this spray nozzle without any particular restriction, but preferably, a spray nozzle capable of realizing the average particle size of mist droplets described above and spraying a small amount of liquid over a wide range is used. Specifically, for example, products called “KB nozzle”, “KBN nozzle” and the like manufactured by H. Ikeuchi & Co., Ltd. can be used. In other words, a nozzle provided with a main body spout and a closer and capable of spraying fine mist in an annular or circular shape can be used, the main body spout and closer being made of ceramic.

The amount of the sanitizer per spray by the spray nozzle 7 is adjusted to achieve the aforementioned deposition amount of 0.2 mg/cm² to 2 mg/cm², and is preferably 10 ml or less, depending on the internal volume of the housing.

As shown in FIG. 2, the spray nozzle 7 is arranged at the center of the insertion space for the object to be sanitized in a width direction (in the direction of arrow A in FIG. 1) so as to protrude downward from a ceiling surface of the space shown in FIG. 2, at the side opposite to the opening 2a configuring one surface of the housing 2b. However, the position for arranging the spray nozzle 7 is not limited to this location; an aspect in which the spray nozzle 7 is provided at the center of the surface opposite to the opening 2a configuring one surface of the housing 2b, or an aspect in which the spray nozzle 7 is provided on each width-wise side of the center in a depth direction (direction of arrow B in FIG. 1), can be adopted. Furthermore, an aspect is possible in which the UV lamp 3 for radiating ultraviolet light may be provided below and the spray nozzle 7 may be provided on the ceiling surface; this aspect is particularly economical.

### Ultraviolet Irradiation Unit

The ultraviolet irradiation unit is configured to be able to irradiate the inside of the insertion space 2 for the object to be sanitized, and the UV lamps 3 above and below the housing 2b are provided at positions away from the opening 2a of the housing 2b, respectively. Accordingly, a structure is formed in which the UV light emitted from the UV lamps 3 is less likely to leak to the outside of the housing 2b. In order to obtain such a structure, the positions of the UV lamps are preferably 5 to 20 cm or more, more preferably 10 to 20 cm or more, away from the opening 2a toward the inside of the housing. Note that the wavelength of the ultraviolet light emitted by the UV lamps 3 is preferably 206 to 208 nm or 221 to 223 nm, and particularly preferably 221 to 223 nm. As a lamp that can be used in this case, a lamp used for the ultraviolet irradiation described above can be appropriately used.

### Insertion Space for Object To Be Sanitized

The housing 2b is in a hexahedral shape, and the inside thereof is configured as the insertion space 2 for the object to be sanitized. The inside of the insertion space 2 for the object to be sanitized is completely sealed except for the spray nozzle 7, and is configured such that the sprayed alcohol-based sanitizer does not adversely affect an electrical system by adhering directly to the UV lamps 3 and other wiring of the electrical system. Specifically, inner wall surfaces of the housing 2b are all sealed so that the UV lamps 3 and an energizer that supplies power to the sanitizer supply unit and the ultraviolet irradiation unit are not exposed in the insertion space 2 for the object to be sanitized. In the installation location of the spray nozzle 7 as well, the periphery of the spray nozzle 7 is sealed with a commonly used sealing material so that the sprayed alcohol-based sanitizer does not ooze out of the housing 2b.

The area of the opening 2a provided on one surface of the housing 2b needs to be large enough to enable the insertion of a hand without having the hand touch the edges of the opening, but should not be excessively large in order to prevent the sanitizer from blowing out from the inside. Therefore, the area is preferably 30 cm² to 500 cm², or more preferably 200 cm² to 450 cm².

Further, in the present embodiment, a convex surface 2c is formed around the opening 2a on one surface provided with the opening 2a. The convex surface 2c functions as a baffle plate against the blowout of the alcohol-based sanitizer, and a small spray quantity of the alcohol-based sanitizer can be deposited onto the entire hand, enabling effective sanitization of the hand. Further, if necessary, the baffle plate may be provided on at least the ceiling surface, a lower surface, or both left and right surfaces of the inner walls of the housing along the direction of arrow A or B shown in FIG. 1.

Furthermore, at least part of the inner walls of the housing is made of a reflective material that reflects ultraviolet light of 222 nm. Specifically, as shown in FIG. 2, inner surfaces of the housing 2b are composed of a reflective surface 2d made of a reflective material and a transparent permeable surface 2e that transmits the ultraviolet light. The permeable surface 2e is provided on a front surface of each UV lamp 3, and all other wall surfaces, including an inner surface of the convex surface 2c, are configured as reflective surfaces. The reflecting surfaces are each preferably made of a material (reflecting mirror) that reflects ultraviolet light of 222 nm, and aluminum, silver, stainless steel or the like is used as the material. Also, as the reflective surfaces, a metal material whose surface is coated with a material having 70% or higher transmittance transmitting ultraviolet light of 222 nm (for example, quartz film or a transparent fluororesin such as ETFE) can be used. As a material constituting the permeable surface 2e, it is preferred to use a material having a transmittance of 222-nm ultraviolet light of 70% or more, preferably 80% or more, such as quartz glass or a transparent fluorine film such as ETFE. As the quartz material, commercially available products such as products “S”, “ES”, “EDA”, “EDH” manufactured by Tosoh Corporation, and SUPRASIL series manufactured by Shinetsu Quartz Co., Ltd. can be used.

By adopting the structure of such device, the desired effects of the present invention described above can be obtained.

### Other Members

During ultraviolet irradiation, an indication showing the irradiation is preferably displayed. As an ultraviolet irradiation display device, an energizing lamp or a fluorescent substance that emits light in response to ultraviolet light of 222 nm is preferably installed.

Although not shown in particular, a sensor for detecting the insertion of a hand is provided in the vicinity of the opening. When this sensor detects the entry of a hand, the spray nozzle sprays the alcohol-based sanitizer 1 to 2 seconds after the detection, and at the same time, ultraviolet light is radiated by the UV lamps

### Usage (the Sanitizing Method Using the Sanitizing Device of the Present Invention)

Next, the sanitizing method using the device 1 of the present embodiment is described.

In the sanitizing method of the present embodiment, by putting a hand into the housing through the opening 2a, a sensor, not shown particularly, reacts to allow the alcohol-based sanitizer to be sprayed from the spray nozzle 7 and at the same time allow ultraviolet light to be emitted from the UV lamps 3.

In the device of the present embodiment, as described above, the spray nozzle is provided at the center of one surface (the center of a front surface, with the hand as a reference) and the UV lamps are provided above and below the hand, and the convex surface and reflective surfaces are installed as well. Therefore, the sanitizer can be sprayed onto the entire hand, enabling the simultaneous execution of disinfection by UV irradiation by the UV lamps 3 and sanitization by spraying the sanitizer by the spray nozzle 7, without inhibiting the effects thereof.

Owing to such configuration, the time it takes to spray can be shortened to 3 seconds or less, or even 1 second or less.

The UV lamps in the hand sanitizing device preferably has an irradiation intensity of 10 mw/cm² or less and a radiation amount of 100 mJ/cm² or less in terms of achieving short-time sanitization and downsizing of the device. A lower limit of the irradiation intensity is approximately 0.1 mw/cm², and a lower limit of the radiation amount is approximately 0.2 mJ/cm². An irradiation time is preferably 15 seconds or less, and more preferably 5 seconds or less. In other words, since the treatment with the alcohol-based sanitizer and the ultraviolet irradiation are performed in combination, sufficient disinfection and sanitization can be achieved even if the irradiation time is shortened and the alcohol-based sanitizer is used in a small amount. In order to achieve said sufficient disinfection and sanitization, as well as to sanitize the hand quickly, reduce harmful effects on the skin such as chapped hands, and further reduce the size of the equipment, the amount of the alcohol-based sanitizer to be sprayed is preferably such that the amount of the alcohol-based sanitizer to be actually deposited onto the hand is preferably 0.1 mg/cm² to 3 mg/cm², and more preferably 0.2 mg/cm² to 2 mg/cm². The amount of ultraviolet radiation is preferably 0.2 mJ/cm² to 10 mJ/cm², and more preferably 0.2 mJ/cm² to 5 mJ/cm². If the amount of the sanitizer used or the amount of ultraviolet radiation is less than the ranges described above, the sanitization will be insufficient or it will take a long time to sanitize. If the amount of the sanitizer used is greater than the corresponding range described above, a higher risk of chapped hands is considered, and if the amount of ultraviolet radiation is higher, the device will be excessively large and more expensive.

Therefore, it is most preferable that the alcohol-based sanitization treatment be executed in such a manner that the amount of the alcohol-based sanitizer to be deposited onto the object to be sanitized is 0.2 mg/cm² to 2 mg/cm², and it is most preferable that the amount of ultraviolet radiation be 0.2 mJ/cm² to 5 mJ/cm².

### Other Aspects

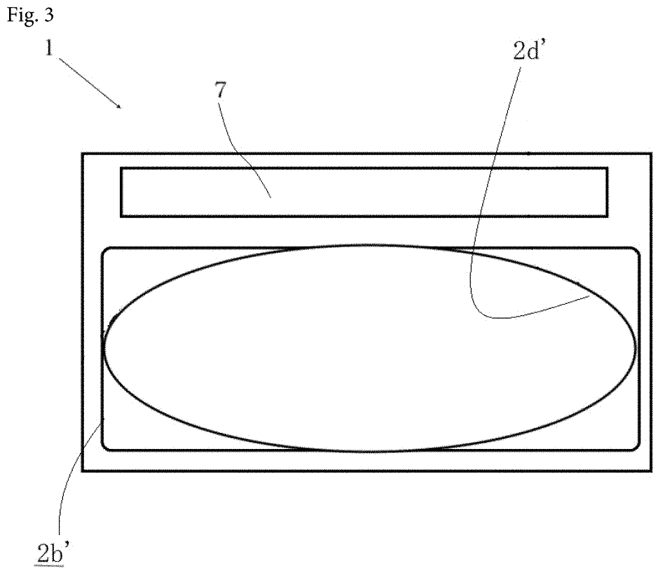

Also, in the device 1 of the present invention, an internal form of the housing 2b can be the form shown in FIG. 3. That is, in the form shown in FIG. 2, the internal form of the housing 2b is configured so as to form a rectangular parallelepiped space. However, as shown in FIG. 3, the reflective surface 2d having an oval cross-sectional shape in the width direction can be formed. By configuring the internal form of the housing 2b in this manner, not only is it possible to maximize the reflection effect of the reflective surface 2d inside the housing 2b, but also a high sanitization effect can be achieved within a shorter period of time.

Moreover, although not particularly illustrated, an auxiliary air ejection nozzle may be installed on the ceiling surface or the lower surface so that a mist of the sanitizer can fly within the space. As a result, the sanitizer can be spread all over the hand, accomplishing a high sanitization effect.

### Effects

For the number of bacteria after a certain short-time sanitization treatment to become 1/100 or less of that obtained prior to the treatment according to an in vivo evaluation method of an hand sanitizer (antiseptic) used in the ASTM E1174 method in relation to hygienic hand hygiene (sanitization) of a healthcare professional, the sanitizing device and sanitizing method of the present embodiment are capable of realizing the number of bacteria within a shorter period of time, with a smaller device, with a smaller amount of sanitizer, and a lower amount of ultraviolet radiation, compared to the prior art.

Note that the sanitizing method and sanitizing device of the present invention are not limited to the foregoing embodiments and can be changed in various ways without departing from the gist of the present invention.

For example, only one UV lamp may be provided, and only the opposing surfaces of said UV lamp can be configured as the reflective surfaces. Further, the reflective surfaces do not have to be flat reflecting mirrors but can be configured as non-planar reflecting mirrors.

The structure of the device is not limited to the foregoing structure. For example, when installing the sanitizing device on the surface of a wall, a structure is possible in which the housing for inserting a hand is provided in an upper part of the device and the opening is provided on an upper surface of the device, to allow the insertion of the hand from top to bottom.

Also, as to the location of the UV lamp, other than the aspect in which the ultraviolet light is emitted in the direction perpendicular to the top surface or bottom surface of the hand as in the foregoing embodiments, various locations are possible so as to emit ultraviolet light in oblique directions.

The above has described that up to two spray nozzles are provided, but three or more spray nozzles may be provided. As to the location of the spray nozzles, although the foregoing aspect described that the sanitizer is sprayed from the fingertips side in a direction parallel to the surfaces of the hand, various locations are possible in which the sanitizer is sprayed onto the hand from the two nozzles in the direction perpendicular to the top surface or bottom surface of the hand, the two nozzles being provided above and below the hand respectively, or the sanitizer is sprayed in oblique directions.

In addition, although the spraying and the radiation are executed automatically in the foregoing example, the spraying and the radiation may be performed manually, and the ultraviolet irradiation can be performed before or after the spraying.

### EXAMPLES

The present invention will be described hereinafter in more detail using experimental examples and comparative examples, but the present invention is not limited thereto.

### Experimental Example 1

A) A nuclepore filter (Whatman, 0.2 μm pore size, φ47 mm, polycarbonate) was used as a model for a hand, and said filter was inoculated with 10 ml of cell fluid prepared by adjusting the precultured Serratia marcescens to approximately 8×10⁶ CFU/ml, and the resultant filter was dried and used as a sample.

B) The sample was inserted into the sanitizing device shown in FIGS. 1 and 2, and, as the alcohol-based sanitizer, Japanese Pharmacopoeia disinfectant ethanol (aqueous solution containing 76.9 to 81.4 vol % ethanol (C2H5H)) was sprayed from the spray nozzle onto the sample for three seconds in such a manner that the amount of the ethanol deposited on the sample was 1.0 mg/cm² (filter area). At the same time as this spraying, a 222 nm excimer lamp (product name “UMK20-22XE” manufactured by Sen Engineering Co., Ltd.) was used to emit ultraviolet light with an irradiation intensity of 0.24 mw/cm² (irradiation energy 1 mJ/cm²) for four seconds. After the irradiation, the sanitized filter was placed in a petri dish containing 10 mL of SCDLP broth culture medium (Eiken). The culture medium was then treated with an ultrasonic wave cleaning machine for two minutes to wash out deposited bacteria, and the number of bacteria was measured using the resultant medium as a sample solution for measuring the number of bacteria. A 10-fold serial dilution was prepared for the sample solution for measuring the number of bacteria by using a physiological saline solution, and the stock solution or the diluted solution was cultured using a pour plate method, to measure the number of viable bacteria (CFU). The culturing was executed at 36±2° C. for 40 to 48 hours.

The sanitization results are shown in Table 1.

### Experimental Example 2

A sanitization experiment was conducted in the same manner as in Experimental Example 1 except that the ultraviolet irradiation in Experimental Example 1 was performed with an irradiation intensity of 0.7 mw/cm² (irradiation energy of 3.5 mJ/cm²) for five seconds. The sanitization results are shown in Table 1.

### Experimental Example 3

A sanitization experiment was conducted in the same manner as in Experimental Example 1 except that the Japanese Pharmacopoeia disinfectant ethanol was sprayed onto the sample in Experimental Example 1 in such a manner that the amount of the ethanol deposited on the sample was 1.8 mg/cm² (filter area) and that the sample was irradiated with ultraviolet light with an irradiation intensity of 0.14 mw/cm² (irradiation energy of 0.56 mJ/cm²) for four seconds. The sanitization results are shown in Table 1.

### Experimental Example 4

A sanitization experiment was conducted in the same manner as in Experimental Example 1 except that the Japanese Pharmacopoeia disinfectant ethanol was sprayed onto the sample in Experimental Example 1 in such a manner that the amount of the ethanol deposited on the sample was 0.5 mg/cm² (filter area). The sanitization results are shown in Table 1.

### Comparative Example 1

A sanitization experiment was conducted in the same manner as in Experimental Example 1 except that the ultraviolet irradiation in Experimental Example 1 was not performed and the Japanese Pharmacopoeia disinfectant ethanol was sprayed onto the sample in such a manner that the amount of the ethanol deposited on the sample was 1.0 mg/cm² (filter area). The sanitization results are shown in Table-1.

Comparative Example 2

A sanitization experiment was conducted in the same manner as in Experimental Example 1 except that the spraying in Experimental Example 1 was not performed and that the sample was irradiated with ultraviolet light with an irradiation intensity of 0.4 mw/cm² (irradiation energy of 2 mJ/cm²) for five seconds. The sanitization results are shown in Table 1.

Experimental Example 5

A sanitization experiment was conducted in the same manner as in Experimental Example 1 except that in Experimental Example 1 the sample was obtained by inoculating the filter with 10 ml of cell fluid prepared by adjusting Staphylococcus aureus (MRSA) to 6.1×10⁴ CFU/ml, the resultant filter was dried and sprayed with the Japanese Pharmacopoeia disinfectant ethanol in such a manner that the amount of the ethanol deposited on the sample was 1 mg/cm² (filter area), and that the sample was irradiated with ultraviolet light with an irradiation intensity of 0.8 mw/cm² (irradiation energy of 4 mJ/cm²) for five seconds. The sanitization results are shown in Table 1.

### Comparative Example 3

A sanitization experiment was conducted in the same manner as in Experimental Example 5 except that the ultraviolet irradiation in Experimental Example 5 was not performed and that the Japanese Pharmacopoeia disinfectant ethanol was sprayed onto the sample in such a manner that the amount of the ethanol deposited on the sample was 1 mg/cm² (filter area). The sanitization results are shown in Table-1.

### Comparative Example 4

A sanitization experiment was conducted in the same manner as in Experimental Example 5 except that the spraying in Experimental Example 5 was not performed and that the sample was irradiated with ultraviolet light with an irradiation intensity of 0.7 mw/cm² (irradiation energy of 2.8 mJ/cm²) for four seconds. The sanitization results are shown in Table-1.

TABLE 1

| | | | | Control | After treatment |
|---|---|---|---|---|---|
| Experiment | | Ethanol | Ultraviolet | Cell count | Cell count |
| No. | Bacteria | (mg/cm2) | (mJ/cm2) | CFU | CFU |
| ex-1 | Serratia | 1 | 0.96 | 5.3 × 10 [symbol] | 50 |
| ex-2 | marcescens | 1 | 3.5 | | 50 |
| ex-3 | | 1.8 | 0.56 | | <10 |
| ex-4 | | 0.5 | 0.96 | | 5.5 × 10 [symbol] |
| coex-1 | | 1 | | | 10 × 10⁵ |
| coex-2 | | — | 2 | | 4.9 × 10⁵ |
| ex-5 | MRSA | 1 | 4 | 2.4 × 10 [symbol] | <1 |
| coex-3 | | 1 | — | 5.7 × 10 [symbol] | 1.9 × 10 [symbol] |
| coex-4 | | — | 2.8 | | 7.1 × 10 [symbol] |

[symbol]

indicates data missing or illegible when filed

As described above, the CDC and WHO guidelines require that approximately 2 to 3 ml of alcohol-based sanitizer be rubbed into hands at a time. In addition, the surface area of ⅓ of the forearms and both hands of a healthy, normal Japanese adult is said to be approximately 1000 cm² (Kurazumi et al. (1994) Japanese journal of meteorology Volume 31 Issue 1, 1994, pp. 5-29.). Therefore, according to the CDC and WHO guidelines, 2×10⁻³ to 3×10⁻³ ml/cm², or approximately 2 to 3 mg/cm² of alcohol-based sanitizer should be rubbed into the hands. However, as a result of evaluating the bactericidal effect of disinfectant ethanol and 222-nm light on Serratia marcescens (Table-1), the number of Serratia marcescens was reduced to approximately 1/600 by ethanol deposition of approximately 1 mg/cm², which is certainly a considerable bactericidal effect (Comparative Example 1). However, by radiating a small amount (approximately 1 mJ/cm²) of 222-nm light along with the spraying of the ethanol, the bactericidal effect has dramatically improved by approximately 1000 times (the number of remaining Serratia marcescens was approximately 1/1,000,000 (Experimental Example 1)). Furthermore, even for MRSA resistant Staphylococcus aureus), which has higher alcohol resistance than Serratia marcescens, the combination of ethanol deposition of 1 mg/cm² and radiation of 222-nm light at 4 mJ/cm² brought about almost perfect bactericidal effect (Experimental Example 5). Accordingly, by radiating 222-nm ultraviolet light along with the spraying of the sanitizer, the bactericidal effect equivalent to the CDC and WHO guidelines can be achieved even when the amount of the alcohol-based sanitizer used is even smaller, which leads to a reduction of chapped hands caused by alcohol-based sanitizers.

When spraying the sanitizer onto the hands using an alcohol-based sanitizer sprayer, if the sprayer is designed in such a manner that approximately 10 to 60% of the sprayed amount is deposited onto the hands, the amount of sanitizer per spray can be approximately 10 ml or less.

1. A sanitizing method comprising: a step of executing both radiation of ultraviolet light having a wavelength of 190 nm to 230 nm and a treatment with an alcohol-based sanitizer.

2. The sanitizing method according to claim 1 above, wherein the alcohol-based sanitizer contains water and an alcoholic agent selected from among ethanol, propanol, and a mixture thereof, the alcoholic agent being blended in a greater amount than the water.

3. The sanitizing method according to claim 1, wherein the ultraviolet light has a wavelength of 206 to 208 nm or 221 to 223 nm.

4. The sanitizing method according to claim 1, comprising a step of executing an alcohol-based sanitizer treatment in which a deposition amount of the alcohol-based sanitizer on an object to be sanitized is 0.2 mg/cm² to 2 mg/cm², and a step of setting an amount of ultraviolet radiation to 0.2 mJ/cm² to 5 mJ/cm².

5. A sanitizing device comprising: a sanitizer supply unit for depositing an alcohol-based sanitizer onto an object to be sanitized, and an ultraviolet irradiation unit for irradiating the object to be sanitized with ultraviolet light having a wavelength of 190 nm to 230 nm.

6. The sanitizing device according to claim 5, wherein the ultraviolet light has a wavelength of 206 to 208 nm or 221 to 223 nm.

7. The sanitizing device according to claim 5, wherein the sanitizer supply unit sprays the alcohol-based sanitizer in an amount at which a deposition amount of the alcohol-based sanitizer on the object to be sanitized is 0.2 mg/cm² to 2 mg/cm², and an amount of ultraviolet radiation is 0.2 mJ/cm² to 5 mJ/cm².

8. The sanitizing device according to claim 5, further comprising an insertion space for the object to be sanitized, wherein the sanitizer supply unit is configured to be capable of spraying the alcohol-based sanitizer into the insertion space for the object to be sanitized, the ultraviolet irradiation unit is configured to be capable of irradiating the inside of the insertion space for the object to be sanitized, the insertion space for the object to be sanitized being composed of a housing, a part of which has an opening, and being configured as an independent space so that the ultraviolet irradiation unit and an energizer that supplies power to the sanitizer supply unit and the ultraviolet irradiation unit are not exposed into the space, preventing components of the alcohol-based sanitizer to be sprayed from entering the ultraviolet irradiation unit and the energizer.

9. The sanitizing device according to claim 8, wherein the opening has an area of 200 cm² to 450 cm².

10. The sanitizing device according to claim 5, wherein at least the insertion space for the object to be sanitized partially has a wall surface made of a reflective material that reflects ultraviolet light having a wavelength of 222 nm.
